(19) United States
(12) Patent Application Publication
(10) Pub. No.: US 2016/0206840 A1
(43) Pub. Date: Jul. 21, 2016

(54) AIRWAY DEVICE WITH MULTIPLE CHANNELS

(71) Applicant: City of Hope, Duarte, CA (US)

(72) Inventor: Walter L. S. Chang, Toluca Lake, CA (US)

(21) Appl. No.: 14/993,907

(22) Filed: Jan. 12, 2016

Related U.S. Application Data
(60) Provisional application No. 62/104,587, filed on Jan. 16, 2015.

(51) Int. Cl. A61M 16/04; A61M 16/08

(52) U.S. Cl. CPC A61M 16/0461; A61M 16/0488; A61M 16/0816; A61M 2230/432

(57) ABSTRACT

A mechanical airway and methods of using the same are disclosed herein. The mechanical airway can include an elongate member having a first end and a second end, and the elongate member can define a central lumen, a first channel, and a second channel. In some embodiments, the central lumen can extend through the elongate member from the first end to the second end. The first channel and the second channel can include first openings at the first end of the elongate member and second openings that connect each of the first and second channels to the elongate member.

### REFERENCE TO PRIORITY DOCUMENT

This application claims priority to co-pending U.S. Provisional Patent Application Ser. No. 62/104,587 entitled “Airway Device With Multiple Channels” and filed on Jan. 16, 2015. Priority to the aforementioned filing date is claims and the provisional patent application is incorporated by reference in its entirety.

### BACKGROUND

A blocked airway presents a significant and urgent medical problem that must be addressed, either via the removal of the blockage from the airway or via the circumventing of the blockage of the airway, to preserve the life of a patient having such a blocked airway. These blockages can arise in many situations, but are frequently the result of trauma, such as facial trauma or trauma to tissue surrounding the airway, or the relaxation of tissue surrounding the airway, as can occur as a result of anesthesia and/or sedation. This trauma or relaxation of tissue surrounding the airway can lead to the collapse of all or portions of the airway, which can block the airway.

Such blockages due to collapsed and/or damaged airways can be addressed and/or prevented through the use of a mechanical airway device. The mechanical airway device can be sized and shaped to fit in a patient's airway, such as one of the nasal and oral passageways, to maintain an open airway. While such mechanical airways provide significant benefits, mechanical airways remain simple devices that are not readily adaptable to modern healthcare.

### SUMMARY

One aspect of the present disclosure relates to a nasopharyngeal airway. The nasopharyngeal airway can include an elongate member having a first end and an opposing second end. In some embodiments, the elongate member defines a central lumen having a longitudinal axis, which central lumen extends from the first end of the elongate member to the opposing second end of the elongate member. In some embodiments, the elongate member can define a first channel adjacent to the central lumen, which first channel includes a first opening at the first end, and a second opening. In some embodiments, the second opening of the first channel fluidly connects the first channel to the central lumen. In some embodiments, the elongate member can define a second channel adjacent to the central lumen, which second channel includes a first opening and a second opening. In some embodiments, the second opening of the second channel fluidly connects the second channel to the central lumen.

In some embodiments of the airway, the second opening of the first channel and the second opening of the second channel are located between the first and second ends of the elongate member. In some embodiments the airway can include a first connector located at the first opening of the first channel, which first connector is fluidly connected to the first channel. In some embodiments of the airway, the first connector can be a luer lock. In some embodiments, the luer lock can be a female luer lock.

In some embodiments the airway can include a first tube having a first end and a second end, which first end of the first tube connects to the first end of the elongate member. In some embodiments, the first tube defines a portion of the second channel. In some embodiments, the airway can include a second connector located at the second end of the first tube.

In some embodiments of the airway, the elongate member is curved. In some embodiments of the airway, the elongate member is made of a deformable material. In some embodiments, the second channel contains an end-tidal CO₂ monitor. In some embodiments, the elongate member is sized and shaped for insertion into a nasal passageway. In some embodiments of the airway, the second end of the elongate member is angled.

One aspect of the present disclosure relates to a method of creating an unobstructed airway. In some embodiments, the method includes inserting a mechanical airway into the airway of a patient to thereby prevent the obstruction of the airway of the patient. The mechanical airway can include an elongate member having a first end and an opposing second end, which elongate member defines a central lumen having a first central axis, which central lumen extends from the first end and to the opposing second end of the elongate member, a first channel adjacent to the central lumen, which first channel includes a first opening at the first end, and a second opening, which second opening of the first channel fluidly connects the first channel to the central lumen, and a second channel adjacent to the central lumen, which second channel includes a first opening and a second opening, which second opening of the second channel fluidly connects the second channel to the central lumen. In some embodiments, the method can include securing the mechanical airway in the airway of the patient.

In some embodiments of the method, the mechanical airway further includes a first connector located at the first opening of the first channel, which first connector is fluidly connected to the first channel. In some embodiments, the mechanical airway further includes a first tube having a first end and a second end, which first end of the first tube connects to the first end of the elongate member. In some embodiments, the first tube defines a portion of the second channel.

In some embodiments of the method, the mechanical airway further includes a second connector located at the second end of the first tube. In some embodiments the method includes fluidly connecting an end-tidal CO₂ monitor to the first channel. In some embodiments, the end-tidal CO₂ monitor is connected to the first channel via the first connector.

In some embodiments, the method includes connecting the second channel to an oxygen source. In some embodiments, the second channel is connected to the oxygen source via the second connector. In some embodiments, the mechanical airway is inserted into a nasal passageway, and in some embodiments, the mechanical airway is inserted into an oral passageway. In some embodiments, the mechanical airway is sized for the patient.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

FIG. 1 is a perspective view of one embodiment of a nasopharyngeal airway.

FIG. 2 is a section view of one embodiment of the nasopharyngeal airway shown in FIG. 1.

FIG. 3 is a section view of a second embodiment of the nasopharyngeal airway shown in FIG. 1.

FIG. 4 is a perspective view of one embodiment of an oral airway.

FIG. 5 is a flowchart illustrating one embodiment of a process for using a mechanical airway as described herein.

In the appended figures, similar components and/or features may have the same reference label. Where the reference label is used in the specification, the description is applicable to any one of the similar components having the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference.

### DETAILED DESCRIPTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

### Definitions

As used herein, an “airway” is a path through which air flows to get into and/or out of the lungs. An airway can include a “nasal passageway,” also referred to herein as a “nasal passage,” and/or an “oral passageway,” also referred to herein as an “oral passage.” As used herein, a “nasal passage” is a channel for airflow through the nose, and as used herein, an “oral passage” is a channel for airflow through the mouth. In some embodiments, the nasal and/or oral passages can include all or portions of the channel for airflow from one or both of the nostrils and/or lips to the pharynx, larynx and/or trachea.

As used herein, a “mechanical airway,” also referred to herein as an “airway adjunct,” is a medical device used to maintain or open a patient's airway. The “mechanical airway” can include a “nasopharyngeal airway,” also referred to herein as a “nasal airway,” and/or an “oropharyngeal airway,” also referred to herein as an “oral airway.” The nasal airway is a tube that is designed to be inserted into the nasal passageway to secure an open airway, and the oral airway is a device that secures an open airway by preventing the tongue and pharyngeal soft tissues from obstructing the laryngeal inlet.

The mechanical airway can be a variety of shapes and sizes. By being made in different sizes, the airways can be used in patients having anatomical structures of different sizes. In some embodiments, the mechanical airways can be made of a variety of materials. In some embodiments, the mechanical airways, and particularly a nasal airway can be made of an elastomeric material and/or deformable such as, for example polyvinyl chloride (PVC), a synthetic rubber such as, for example, polychloroprene, latex, or the like, and in some embodiments, the mechanical airways, and particularly an oral airway can be made of a rigid and/or semi-rigid material such as, for example polyethylene, polypropylene, or the like.

The mechanical airway can include an “elongate member.” As used herein, an “elongate member” is a portion of an airway having a first end, an opposing second end, and a longitudinal axis extending there between. In some embodiments, the longitudinal axis of the elongate member can be parallel to and/or follow the path of the airway through which the mechanical airway is inserted and/or which the mechanical airway secures. The elongate member can be straight, curved, or a combination of straight and curved.

The mechanical airway can include a “central lumen.” As used herein, a “central lumen” refers to a cavity within the mechanical airway, and specifically within the elongate member, that can, in some embodiments, extend from and/or through the first end and/or second end of the elongate member. In such an embodiments, the central lumen can include a first opening at the first end of the elongate member, a second opening at the second end of the elongate member, and a hollow body portion extending there between. Thus, in some embodiments, the central lumen is bounded and/or defined by portions of the elongate member.

The mechanical airway can include a plurality of “channels.” As used herein, a “channel” refers to a passage extending through the elongate member. In some embodiments, the channel can extend from and/or through the first end of the elongate member to the second end of the elongate member, and in some embodiments, the channel can extend a portion of the distance from the first end of the elongate member to the second end of the elongate member. In some embodiments, the channel can include a first opening that can be, for example, located at the first end of the elongate member, and in some embodiments, the channel can include a second opening that can be, for example, located at the second end of the elongate member, or a position intermediate between the first end and the second end of the elongate member. In some embodiments in which the second opening is located at a position intermediate between the first end and the second end, the second opening can connect to the central lumen such that the channel and the central lumen are fluidly connected.

In some embodiments, the mechanical airway can include a plurality of passages which can include, for example, the central lumen and one or several channels. As used herein, a “multi-channel airway” is a mechanical airway having a plurality of passages.

The one or several channels of the elongate member can include one or several “connectors.” As used herein, a “connector” is a feature that is associated with a channel and that can fluidly and/or mechanically connect a second feature to the associated channel via a mating connector. The connector can connect with a mating connector via any desired mechanism or method. In some embodiments, for example, the connector can be a male or a female luer lock, a male or female connector, a magnet, an adhesive, or the like.

As used herein, an “oxygen source” can be any source of a gas and/or gas/fluid mixture. This can include, for example, oxygen, a combination of oxygen and one or several other gases, or the like. The oxygen source can be, for example, a pressurized vessel, or an outlet of an oxygen delivery system as is found in, for example, a hospital or a medical facility.

As used herein, “approximately” identifies a range that is approximately 1%, 5%, 10%, 15%, 20%, and/or 25% of the therewith associated value, dimension, and/or parameter.

### Multi-Channel Airway

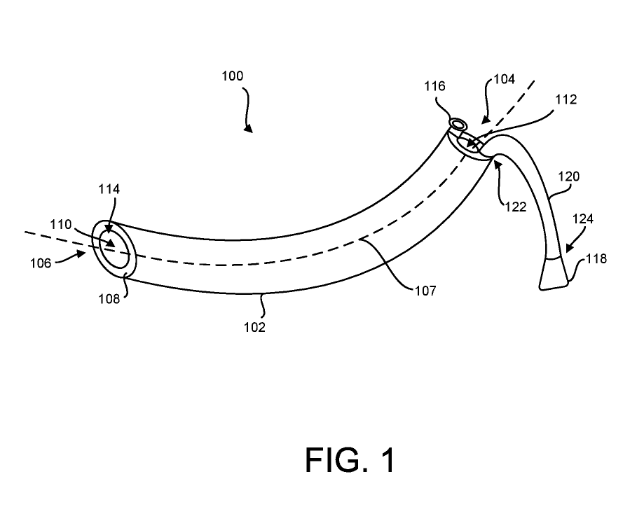

With reference now to FIG. 1, a perspective view of one embodiment of a multi-channel airway 100 is shown, which, in some embodiments, can be a nasopharyngeal airway. The multi-channel airway 100 depicted in FIG. 1 includes an elongate member 102 having a first end 104, a second end 106, and a longitudinal axis 107 extending there between. The elongate member 102 can have a wall 108 that defines a central lumen 110 and channels that will be discussed below.

The elongate member 102 can comprise a variety of shapes and sizes. In some embodiments, a cross-section of the elongate member 102, and specifically the external perimeter of a cross-section of the wall 108, taken on a plane perpendicular to the longitudinal axis can have a circular shape, a rectangular shape, a triangular shape, a pentagonal shape, a hexagonal shape, an octagonal shape, an ovular shape, and/or any other desired shape. In some embodiments, all or portions of the elongate member 102 can be straight, curved, and/or a combination of straight and curved.

In some embodiments, one or both of the first end 104 and the second end 106 can tapered, angled, and/or rounded. This shaping of one or both of the first and second ends 104, 106 can facilitate inserting the mechanical airway 100 into a patient. In the embodiment depicted in FIG. 1, the second end 106 is angled in that the second end 106 does not terminate in one plane that is perpendicular to the longitudinal axis 107.

The elongate member 102 can be made of a variety of materials. In some embodiments, the material of the elongate member 102 and/or of the mechanical airway 100 can be a biocompatible material, and can be rigid or flexible.

The central lumen 110 of the elongate member 102 can include a first opening 112 at the first end 104 and a second opening 114 at the second end 106. The first and second openings 112, 114 of the central lumen 110 can be connected by a body of the central lumen. Thus, in the embodiment depicted in FIG. 1, the central lumen extend from the first end 104 to the second end 106 and extends through the first end 104 and the second end 106 in that the first opening 112 of the central lumen 110 is located in the first end 104 and the second opening 114 of the central lumen 110 is located in the second end 106.

The mechanical airway 100 includes a first connector 116 and a second connector 118. The first connector 116 and the second connector 118 can be any desired type of connector including, for example, a male connector, a female connector, a male or female luer lock, or the like. In the embodiment depicted in FIG. 1, the first connector 116 attaches to the first end 104 of the elongate member 102, and the second connector 118 attaches to a first tube 120 having a first end 122 connected to the first end 104 of the elongate member 102 and a second end 124 attaching to the second connector 118.

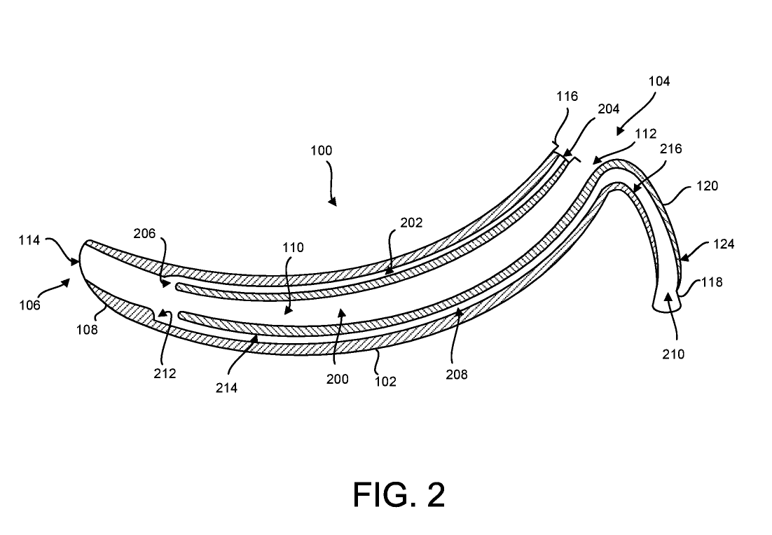

With reference now to FIG. 2, a section view of one embodiment of the multi-channel airway 100 is shown. The multi-channel airway 100 includes the elongate member 102 having the first end 104, the second end 106, and the wall 108 defining the central lumen 110 that extends from the first opening 112 in the first end 104 to the second opening 114 in the second end 106. The central lumen 110 includes a body 200 connecting the first and second openings 112, 114. The multi-channel airway 100 further includes the first and second connectors 116, 118 and the first tube 120.

As further seen in FIG. 2, the multi-channel airway 100 includes a first channel 202 having a first opening 204 at the first end 104 and a second opening 206. The first opening 204 of the first channel 202 can be positioned so as to fluidly connect with the first connector 116 and/or so as to fluidly connect with a feature engaged by the first connector 116. The second opening 206 can be located at a variety of positions including, for example, at the second end 106, proximate to the second end 106, and at a position intermediate between the first end 104 and the second end 106. In the embodiment depicted in FIG. 2, the second opening 206 of the first channel 202 is located at a position intermediate between the first end 104 and the second end 106, and terminates into the central lumen 110 and thereby fluidly connects the first channel 202 and the central lumen 110.

The multi-channel airway 100 includes a second channel 208 having a first opening 210 and a second opening 212. The first opening 210 of the second channel 208 can be located at a variety of positions including, for example, at the first end 104 of the elongate member 102 or at the second end 124 of the first tube 120. The first opening 210 of the second channel 208 can be positioned so as to fluidly connect with the second connector 118 and/or so as to fluidly connect with a feature engaged by the second connector 118. The second opening 212 can likewise be located at a variety of positions including, for example, at the second end 106, proximate to the second end 106, and at a position intermediate between the first end 104 and the second end 106. In the embodiment depicted in FIG. 2, the second opening 212 of the second channel 208 is located at a position intermediate between the first end 104 and the second end 106, and terminates into the central lumen 110 and thereby fluidly connects the second channel 208 and the central lumen 110.

In embodiments some embodiments, and as depicted in FIG. 2, the second channel 208 can include a first portion 214 located in the elongate member and a second portion 216 located in the first tube 120. The first and second portions 214, 216 can be fluidly connected so that, for example, a gas can pass from the first opening 210 of the second channel 208 through the first and second portions 214, 216, and out the second opening 212 of the second channel 208.

The addition of the first and second channels 202, 208 can increase the utility of the mechanical airway 100 by allowing integrated use of the mechanical airway 100 with other equipment, systems, and/or components. Specifically, one or several of the first and second channels 202, 208 and the central lumen 110 can be used to deliver one or several medications, treatments, liquids, emulsions, gases, and/or the like to the patient, and/or to provide patient access for one or several sensing and/or measuring devices and/or systems. This can decrease the burden on the doctor in providing medical care by simplifying patient care procedures and decreasing treatment risks.

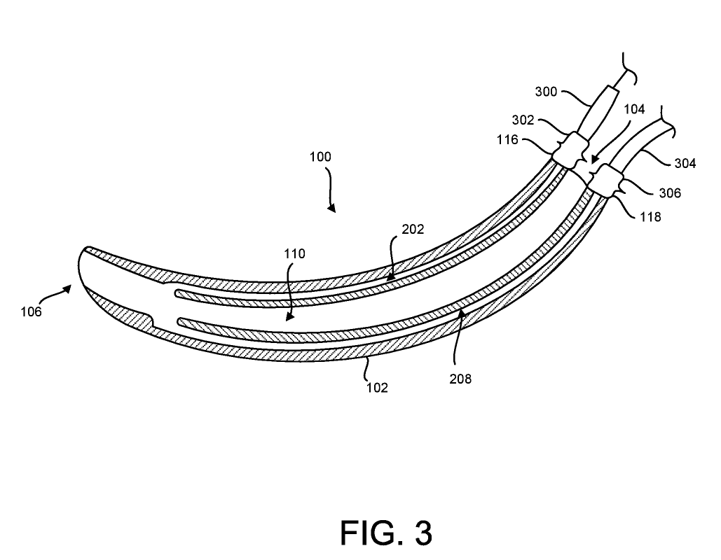

With reference now to FIG. 3, a section view of a second embodiment of the multi-channel airway 100 is shown. In contrast to the embodiment of the multi-channel airway 100 of FIG. 2, the embodiment of the multi-channel airway 100 in FIG. 3 does not include the first tube 120, and the second connector 118 located at the first end 104 of the elongate member 102.

In some embodiments, the second openings 206, 212 of the first and second channels 202, 208 can be located at the same position with respect to one or both of the first or second ends 104, 106 so as to be in the same plane taken perpendicular to the longitudinal axis 107, and in some embodiments, the first and second openings 206, 212 of the first and second channels 202, 208 can be located at different positions with respect to one or both of the first or second ends 104, 106 so as to be in different planes taken perpendicular to the longitudinal axis 107.

As seen in FIG. 3, the first connector 116 of the mechanical airway 100 can be connected to an end-tidal CO₂ monitor 300 via connector 302, and the second connector 118 of the mechanical airway can be connected to an oxygen source 304 via connector 306. The end-tidal CO₂ monitor 300 can be configured to measure the amount and/or concentration CO₂ exhaled by the patient. In some embodiments, the end-tidal CO₂ monitor 300 can be connected to the first channel 202 via the first connector 116 as shown in FIG. 3, can be connected to the second connector 118 located at the end of tube 120, and in some embodiments, the end-tidal CO₂ monitor 300 can be located in the first channel 202.

As discussed above, in some embodiments, the second openings 206, 212 can be in the same plane taken perpendicular to the longitudinal axis 107 or can be in different planes taken perpendicular to the longitudinal axis 107. In some embodiments, the positioning of the second openings 206, 212 with respect to each other can affect performance, and specifically, in some embodiments, the performance of the end-tidal CO₂ monitor 300 can be affected by the relative positioning of the second openings 206, 212. In one particular embodiment, the performance of the end-tidal CO₂ monitor can be improved by placing the second opening 206 of the first channel 202 relatively more proximate to the second end 106 of the elongate member 102 than the second opening 212 of the second channel 208.

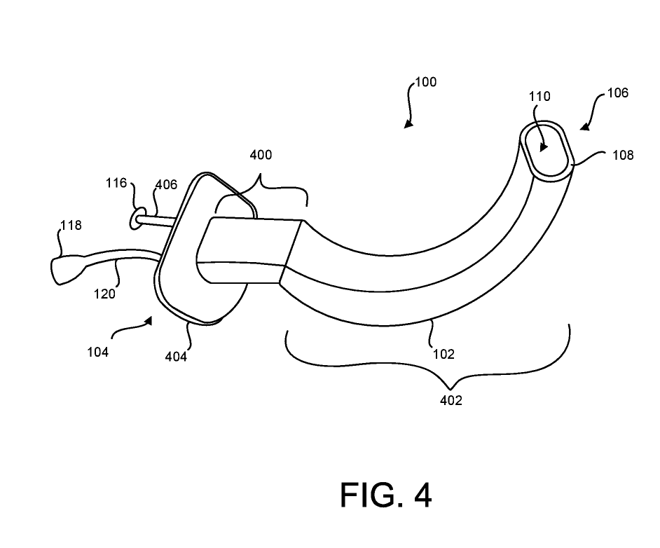

With reference now to FIG. 4, a perspective view of one embodiment of the mechanical airway 100 is shown, which can be, for example, an oral airway. The mechanical airway 100 includes the elongate body 102 having the first end 104 and the second end 106. The elongate body 102 depicted in FIG. 4 further includes a first, straight portion 400 and a second, curved portion 402. In some embodiments, the combination of the first and second portions 400, 402 of the elongate member 102 can facilitate in the placement of the mechanical airway 100 in the oral passageway of the patient.

The elongate member 102 depicted in FIG. 4 further includes a securement feature 404 that can be, for example, a flange. While not shown in FIGS. 1 through 3, some embodiments of the mechanical airway 100 can include a securement feature 404 such as a flange.

In some embodiments, the securement feature 404 can be sized and shaped to prevent the over-insertion of the mechanical airway 100 into the oral passageway of the patient. Additionally, in some embodiments, the securement feature 404 can be used to tape and/or otherwise attach the mechanical airway 100 to the patient to maintain the mechanical airway 100 in a desired position and/or orientation. Although the securement feature 404 is not shown in the embodiments of FIGS. 1 through 3, a person of skill in the art will recognize that the securement feature 404 can be incorporated in those embodiments.

The elongate member 102 defines the central lumen 110 which extends from and/or through the first and second ends 104, 106 of the elongate member 102. Although not shown, the elongate member 102 additionally includes the first and second channels as depicted in FIGS. 1 through 3.

Similar to the embodiments of the mechanical airway 100 shown in FIGS. 1 through 3, the first channel in the embodiment of the mechanical airway 100 in FIG. 4 is connected to the first connector 116. However, in the embodiment shown in FIG. 4, the first connector 116 is not located at the first end 104 of the elongate member 102, but rather connects to a second tube 406 that connects to the first end 104 of the elongate member 102. Also similar to the embodiments of the mechanical airway 100 shown in FIGS. 1 through 3, the second channel in the embodiment of the mechanical airway 100 shown in FIG. 4 connects to second connector 118, and specifically, the second connector 118 is connected to the second channel via first tube 120.

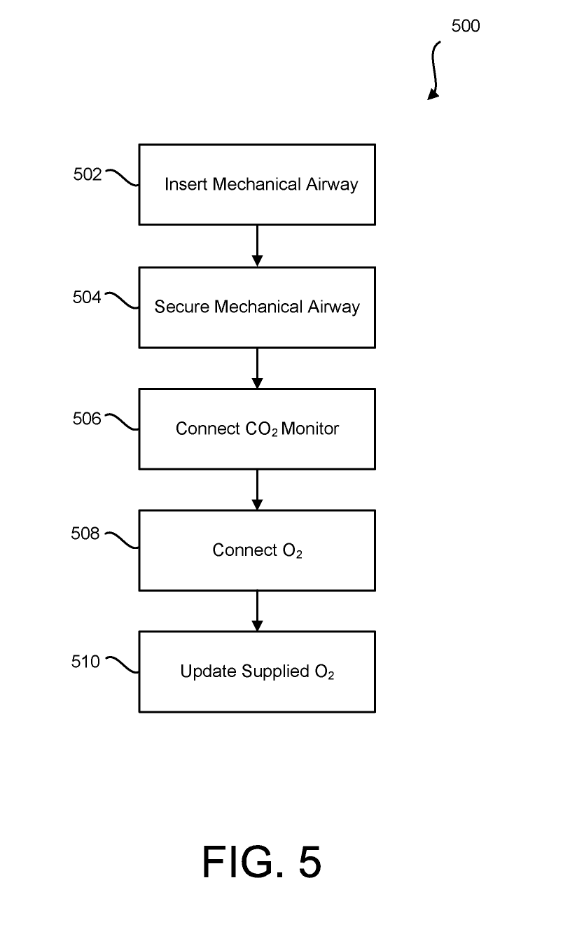

With reference now to FIG. 5, a flowchart illustrating one embodiment of a process 500 for placing a mechanical airway 100 in a patient to prevent the collapsing and/or the obstruction of the patient's airway. The process 500 begins at block 502, wherein the mechanical airway 100 is inserted into the patient's airway such as, for example, inserting the mechanical airway 100 into the nasal passage and/or the oral passage. In some embodiments, the insertion of the mechanical airway 100 into the patient's airway can include selecting a identifying into which patient passage to insert the mechanical airway 100 and sizing the mechanical airway 100 for the patient. In some embodiments, sizing the mechanical airway 100 can include estimating the size of the patient's passage into which the mechanical airway 100 will be inserted, and selected the mechanical airway 100 having a size matching the estimated size of the patient's passage.

After the mechanical airway 100 has been inserted into the patient's airway, the process 500 proceeds to block 504, wherein the mechanical airway 100 is secured. In some embodiments, the mechanical airway 100 can be secured by, for example, taping portions of the mechanical airway 100 such as, for example, the securement feature 404, to the patient.

After the mechanical airway 100 has been secured with respect to the patient, the process 500 proceeds to block 506, wherein the first channel 202 is connected to the end-tidal CO₂ monitor 300. In some embodiments, the first channel 202 can be connected to the end-tidal CO₂ monitor 300 via the mating connection of the first connector 116 and the connector 302 of the end-tidal CO₂ monitor 300. In some embodiments, this mating connection of the first connector 116 and the connector 302 of the end-tidal CO₂ monitor 300 can fluidly connect the first channel 202 to the end-tidal CO₂ monitor 300.

After the first channel 202 has been connected to the end-tidal CO₂ monitor 300, the process 500 proceeds to block 508, wherein the second channel 208 is connected to the oxygen source 304. In some embodiments, the second channel 208 can be connected to the oxygen source 304 via the mating connection of the second connector 118 and the connector 306 of the oxygen source 304. In some embodiments, this mating connection of the second connector 118 and the connector 306 of the oxygen source 304 can fluidly connect the second channel 208 to the oxygen source 304.

After the second channel 208 has been connected to the oxygen source 304, the process 500 proceeds to block 510 wherein data from the end-tidal CO₂ monitor 300 is gathered and the amount of oxygen supplied to the patient via the second channel 208 is varied according to the gathered data from the end-tidal CO₂ monitor 300.

A number of variations and modifications of the disclosed embodiments can also be used. Specific details are given in the above description to provide a thorough understanding of the embodiments. However, it is understood that the embodiments may be practiced without these specific details. For example, well-known circuits, processes, algorithms, structures, and techniques may be shown without unnecessary detail in order to avoid obscuring the embodiments.

Also, it is noted that the embodiments may be described as a process which is depicted as a flowchart, or a block diagram. Although a depiction may describe the operations as a sequential process, many of the operations can be performed in parallel or concurrently. In addition, the order of the operations may be re-arranged. A process is terminated when its operations are completed, but could have additional steps not included in the figure.

While the principles of the disclosure have been described above in connection with specific apparatuses and methods, it is to be clearly understood that this description is made only by way of example and not as limitation on the scope of the disclosure.

What is claimed is:

1. A nasopharyngeal airway comprising:
an elongate member having a first end and an opposing second end, wherein the elongate member defines:
a central lumen having a first central axis, wherein the central lumen extends from the first end and to the opposing second end of the elongate member;
a first channel adjacent to the central lumen, wherein the first channel comprises a first opening at the first end, and a second opening, wherein the second opening of the first channel fluidly connects the first channel to the central lumen;
a second channel adjacent to the central lumen, wherein the second channel comprises a first opening and a second opening, wherein the second opening of the second channel fluidly connects the second channel to the central lumen.

2. The nasopharyngeal airway of claim 1, wherein the second opening of the first channel and the second opening of the second channel are located between the first and second ends of the elongate member.

3. The nasopharyngeal airway of claim 1, further comprising a first connector located at the first opening of the first channel, wherein the first connector is fluidly connected to the first channel.

4. The nasopharyngeal airway of claim 3, wherein the first connector comprises a luer lock.

5. The nasopharyngeal airway of claim 4, wherein the luer lock comprises a female luer lock.

6. The nasopharyngeal airway of claim 1, further comprising a first tube having a first end and a second end, wherein the first end of the first tube connects to the first end of the elongate member.

7. The nasopharyngeal airway of claim 6, wherein the first tube defines a portion of the second channel.

8. The nasopharyngeal airway of claim 7, further comprising a second connector located at the second end of the first tube.

9. The nasopharyngeal airway of claim 1, wherein the elongate member is curved.

10. The nasopharyngeal airway of claim 9, wherein the elongate member comprises a deformable material.

11. The nasopharyngeal airway of claim 1, wherein the second channel contains an end-tidal CO₂ monitor.

12. The nasopharyngeal airway of claim 1, wherein the elongate member is sized and shaped for insertion into a nasal passageway.

13. The nasopharyngeal airway of claim 1, wherein the second end of the elongate member is angled.

14. A method of creating an unobstructed airway comprising:
inserting a mechanical airway into the airway of a patient to thereby prevent the obstruction of the airway of the patient, the mechanical airway comprising:
an elongate member having a first end and an opposing second end, wherein the elongate member defines:
a central lumen having a first central axis, wherein the central lumen extends from the first end and to the opposing second end of the elongate member;
a first channel adjacent to the central lumen, wherein the first channel comprises a first opening at the first end, and a second opening, wherein the second opening of the first channel fluidly connects the first channel to the central lumen;
a second channel adjacent to the central lumen, wherein the second channel comprises a first opening and a second opening, wherein the second opening of the second channel fluidly connects the second channel to the central lumen; and
securing the mechanical airway in the airway of the patient.

15. The method of claim 14, wherein the mechanical airway further comprises a first connector located at the first opening of the first channel, and wherein the first connector is fluidly connected to the first channel.

16. The method of claim 15, wherein the mechanical airway further comprises a first tube having a first end and a second end, wherein the first end of the first tube connects to the first end of the elongate member.

17. The method of claim 16, wherein the first tube defines a portion of the second channel.

18. The method of claim 17, wherein the mechanical airway further comprises a second connector located at the second end of the first tube.

19. The method of claim 18, further comprising fluidly connecting an end-tidal CO₂ monitor to the first channel.

20. The method of claim 19, wherein the end-tidal CO₂ monitor is connected to the first channel via the first connector.

21. The method of claim 20, further comprising connecting the second channel to an oxygen source.

22. The method of claim 21, wherein the second channel is connected to the oxygen source via the second connector.

23. The method 22, wherein the mechanical airway is inserted into a nasal passageway.

24. The method of claim 23, further comprising sizing the mechanical airway for the patient.

25. The method of claim 22, wherein the mechanical airway is inserted into an oral passageway.

26. The method of claim 25, further comprising sizing the mechanical airway for the patient.
